(19) United States
(12) Patent Application Publication
(10) Pub. No.: US 2018/0059114 A1
(43) Pub. Date: Mar. 1, 2018

(54) MAGNETIC NANOSTRUCTURE FOR DETECTING AND ISOLATING CIRCULATING TUMOR CELLS COMPRISING ANTIBODY- AND MAGNETIC NANOPARTICLE-CONJUGATED CONDUCTIVE POLYMER

(71) Applicant: NATIONAL CANCER CENTER, Goyang-si (KR)

(72) Inventors: Youngnam CHO, Goyang-si (KR); Eun Sook LEE, Goyang-si (KR); Wooyoung HONG, Goyang-si (KR)

(73) Assignee: NATIONAL CANCER CENTER, Goyang-si (KR)

(21) Appl. No.: 15/246,615

(22) Filed: Aug. 25, 2016

(30) Foreign Application Priority Data
Aug. 24, 2016 (KR) 10-2016-0107979

(51) Int. Cl. G01N 33/574; C12Q 1/68

(52) U.S. Cl. CPC G01N 33/57492; C12Y 111/01007; C12Q 2600/118; G01N 2333/908; C12Q 2600/156; C12Q 1/6886

(57) ABSTRACT

Disclosed is a magnetic nanostructure for detecting and isolating circulating tumor cells including a conductive polymer to which an antibody and magnetic nanoparticles are bound, which enables circulating tumor cells from early cancer patients and various circulating tumor cell types to be effectively detected using a small amount of blood, circulating tumor cells to be monitored with the naked eye through colorimetric detection, and a very small amount of circulating tumor cells present in blood to be efficiently captured with a strong magnetic field generated by a large amount of the loaded magnetic nanoparticles; in detecting, isolating, and collecting the circulating tumor cells in a very small amount, a long nanowire structure and various antibody types are used, whereby contact with cancer cells may be increased and strong bonding may be formed; sensitivity is increased and various interactions with cancer cells are facilitated, thereby exhibiting increased detection and isolation effects.

### CROSS-REFERENCE TO RELATED APPLICATION

This application claims priority to and the benefit of Korean Patent Application No. 2016-0107979, filed on Aug. 24, 2016, the disclosure of which is incorporated herein by reference in its entirety.

The present invention was undertaken with the support of No. 1510070 and No. 1611170 grant funded by a National Cancer Center, from the Ministry of Health and Welfare, the Republic of Korea.

### BACKGROUND

### 1. Field of the Invention

The present invention relates to a method of detecting and isolating circulating tumor cells using an antibody- and magnetic nanoparticle-conjugated conductive nanostructure polymer.

### 2. Discussion of Related Art

Recently, the importance of early cancer diagnosis has greatly risen in prominence. Accordingly, research into early diagnosis methods for cancer is increasing.

However, current cancer diagnosis methods are mainly invasive methods involving collection of tissue samples and endoscopy. Accordingly, liquid biopsy, as an alternative to conventional invasive diagnosis and examination methods, has attracted attention. Liquid biopsy is a non-invasive method in which cancer cell-derived DNA present in blood of each body region is analyzed and thus detailed observation regarding cancer development, metastasis, and the like can be accomplished, simply by examining bodily fluids, such as blood. Accordingly, application of the technique to cancer diagnosis technology has been attempted due to rapid development of genome analysis technology and advantages thereof such as cost reduction.

Meanwhile, circulating tumor cells (CTCs) are detected in a process in which some tumor cells are detached from primary tumors and are introduced into blood vessels or lymphatic vessels, thereby migrating to other tissues or organs. Although it was not completely established at which stage tumors release circulating tumor cells into the bloodstream, it is assumed that it depends upon the type, size, and/or aggressiveness of tumors.

Accordingly, circulating tumor cells (CTCs) are closely related to the diagnosis of cancer and, therefore, a method of isolating and detecting circulating tumor cells from various tumors derived from solid organs and understanding the characteristics thereof has attracted attention. The greatest advantage of a diagnosis method using circulating tumor cells is that circulating tumor cells can be non-invasively, nonoperatively detected using blood, whereby the diagnosis of cancer and prognosis determination for a cancer patient can be made.

However, in advanced cancer, 1 million or more white blood cells are present per ml of blood, whereas there are 10 to 100 circulating tumor cells, i.e., circulating tumor cells are present at a very low concentration. Accordingly, the number of circulating tumor cells (CTCs) present in blood is very small compared to that of white blood cells or platelets therein. In addition, since most of the CTCs are destroyed during circulation in the bloodstream, there are difficulties in detecting CTCs (Korean Patent Application Publication No. 10-2014-0098334).

Therefore, there is a need for technology to address a problem of conventional circulating tumor cell detection technology and thus increase detection efficiency.

### SUMMARY OF THE INVENTION

The present inventors manufactured a magnetic nanostructure for detecting and isolating circulating tumor cells comprising a conductive polymer, to which an antibody and magnetic nanoparticles are bound, and confirmed that, by using the structure, circulating tumor cells from an early-stage cancer patient and various circulating tumor cell types can be effectively detected using a small amount of blood, and circulating tumor cells can be monitored with the naked eye through colorimetric detection. In addition, the present inventors confirmed that the nanostructure has a remarkably increased effect on detection, isolation, and collection of a very small amount of circulating tumor cells in blood. Based on these effects, the present invention was completed.

Therefore, the present invention has been made in view of the above problems, and it is an object of the present invention to provide a magnetic nanostructure for detecting and isolating circulating tumor cells comprising a conductive polymer to which an antibody and magnetic nanoparticles are bound.

It is another object of the present invention to provide a method of detecting and/or isolating circulating tumor cells using the magnetic nanostructure for detecting and isolating circulating tumor cells comprising the conductive polymer to which an antibody and magnetic nanoparticles are bound.

It is still another object of the present invention to provide a diagnosis kit comprising the magnetic nanostructure for detecting and isolating circulating tumor cells comprising the conductive polymer to which an antibody and magnetic nanoparticles are bound.

It is yet another object of the present invention to provide a method of providing information for diagnosing the onset and/or prognosis of cancer using the magnetic nanostructure for detecting and isolating circulating tumor cells comprising the conductive polymer to which an antibody and magnetic nanoparticles are bound.

It is yet another object of the present invention to provide a method of diagnosing cancer using the magnetic nanostructure for detecting and isolating circulating tumor cells comprising the conductive polymer to which an antibody and magnetic nanoparticles are bound.

It will be understood that technical problems of the present invention are not limited to the aforementioned problems and other technical problems not referred to herein will be clearly understood by those skilled in the art from the disclosure below.

In accordance with the present invention, the above and other objects can be accomplished by the provision of a nanostructure for detecting and isolating circulating tumor cells comprising a conductive polymer.

In addition, an antibody may be bound to the conductive polymer and magnetic nanoparticles may be loaded onto the conductive polymer.

In an embodiment of the present invention, the antibody may be one or more selected from the group consisting of anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

In another embodiment of the present invention, the antibody may be an antibody mixture including anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

In still another embodiment of the present invention, the antibody mixture may further comprising horseradish peroxidase (HRP).

In yet another embodiment of the present invention, the conductive polymer may be polyacetylene, polypyrrole, polythiophene, poly(3,4-ethylenedioxythiophene) (PEDOT) polyaniline, or a derivative thereof.

In yet another embodiment of the present invention, the nanostructure may be a nanowire, nanorod, or nanoparticle.

In yet another embodiment of the present invention, the nanowire may have a diameter of 100 nm to 300 nm depending upon a pore size of a used anodic alumina oxide (AAO) template.

In yet another embodiment of the present invention, the nanowire may have a length of 5 μm to 30 μm and an average length of 17 μm.

In yet another embodiment of the present invention, the circulating tumor cells may be circulating tumor cells (CTCs) or circulating tumor stem cells (CTSCs). Preferably, the circulating tumor cells may be circulating tumor cells.

In accordance with an aspect of the present invention, the above and other objects can be accomplished by the provision of a method of detecting and isolating circulating tumor cells, the method including: (1) a step of treating a subject sample with the nanostructure of the present invention; and (2) a step of detecting the circulating tumor cells from the nanostructure using a magnetic field generated by a magnet.

In an embodiment of the present invention, the method of detecting and isolating circulating tumor cells may further include a step of isolating the circulating tumor cells from the nanostructure using a compound. Here, the compound may be glutathione and the sample may be blood.

In accordance with another aspect of the present invention, there is provided a method of colorimetrically detecting circulating tumor cells, the method including: (1) a step of treating a subject sample with the nanostructure according to the present invention, wherein the nanostructure further includes horseradish peroxidase (HRP); and (2) a step of determining a color of the nanostructure with the naked eye.

In an embodiment of the present invention, the method of colorimetrically detecting circulating tumor cells may further include a step of quantifying a concentration of circulating tumor cells in the subject sample by measuring a change in the color of the nanostructure by means of a spectrometer or colorimeter. Here, the sample may be blood.

In accordance with still another aspect of the present invention, there is provided a kit for diagnosing cancer, the kit including the nanostructure for detecting and isolating circulating tumor cells.

In an embodiment of the present invention, the kit may be a biosensor.

In accordance with yet another aspect of the present invention, there is provided a method of providing information for diagnosing the onset and/or prognosis of cancer, the method including a step of extracting or isolating DNA from circulating tumor cells detected with the nanostructure according to the present invention and analyzing the DNA.

In accordance with yet another aspect of the present invention, there is provided a method of diagnosing cancer using the nanostructure according to the present invention.

In accordance with yet another aspect of the present invention, there is provided a composition for detecting and isolating circulating tumor cells, the composition including the nanostructure according to the present invention.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1a illustrates a scanning electron microscope image of antibody mixture-bound polypyrrole magnetic nanoparticles (Ab mixture_mPpyNPs) according to the present invention;

FIG. 1b schematically illustrates a method of detecting and isolating circulating tumor cells using antibody mixture-bound polypyrrole magnetic nanowires (Ab mixture_mPpyNWs) according to the present invention;

FIG. 1c illustrates a scanning electron microscope image of antibody mixture-bound polypyrrole magnetic nanowires (Ab mixture_mPpyNWs) according to the present invention;

FIG. 1d illustrates an average length distribution of antibody mixture-bound polypyrrole magnetic nanowires (Ab mixture_mPpyNWs) according to the present invention;

FIG. 1e illustrates a transmission electron microscope image of an antibody mixture-bound polypyrrole magnetic nanowire (Ab mixture_mPpyNWs) according to the present invention;

FIG. 1f illustrates transverse relaxation rates of antibody mixture-bound polypyrrole magnetic nanowires (Ab mixture_mPpyNWs) according to the present invention and magnetic nanoparticles (MNPs);

FIG. 1g illustrates a magnetic hysteresis loop of antibody mixture-bound polypyrrole magnetic nanowires (Ab mixture_mPpyNWs) according to the present invention and magnetic nanoparticles (MNPs);

FIG. 2a illustrates a cell capture efficiency comparison result between antibody mixture-bound polypyrrole magnetic nanowires (Ab mixture_mPpyNWs) according to the present invention and nanowires (EpCAM_mPpyNWs) using a single antibody;

FIG. 2b illustrates cell capture efficiencies of antibody mixture-bound polypyrrole magnetic nanowires (Ab mixture_mPpyNWs) according to the present invention with various numbers of blood spiked target cells;

FIG. 2c illustrates capture efficiency comparison results of antibody mixture-bound polypyrrole magnetic nanowires (Ab mixture_mPpyNWs) according to the present invention to determine an optimal nanowire concentration;

FIG. 2d illustrates cell capture of antibody mixture-bound polypyrrole magnetic nanowires (Ab mixture_mPpyNWs) according to the present invention;

FIG. 2e illustrates a cell capture efficiency comparison result between antibody mixture-bound polypyrrole magnetic nanoparticles (Ab mixture_mPpyNPs) according to the present invention and magnetic nanowires (Ab mixture_mPpyNWs);

FIG. 3a illustrates quantification results of circulating tumor cells isolated from blood from early-stage breast cancer patients using antibody mixture-bound polypyrrole magnetic nanowires (Ab mixture_mPpyNWs) according to the present invention;

FIG. 3b illustrates immunofluorescent images of circulating tumor cells isolated from blood of early-stage breast cancer patients using antibody mixture-bound polypyrrole magnetic nanowires (Ab mixture_mPpyNWs) according to the present invention;

FIG. 3c illustrates immunohistochemical staining results of circulating tumor cells isolated from blood of early-stage breast cancer patients using antibody mixture-bound polypyrrole magnetic nanowires (Ab mixture_mPpyNWs) according to the present invention;

FIG. 3d illustrates scanning electron microscope images of circulating tumor cells isolated from blood of early-stage breast cancer patients using antibody mixture-bound polypyrrole magnetic nanowires (Ab mixture_mPpyNWs) according to the present invention;

FIGS. 4a and 4b illustrate glutathione-mediated retrieval results of circulating tumor cells captured by compound antibody mixture-bound polypyrrole magnetic nanowires (Ab mixture_mPpyNWs) according to the present invention;

FIG. 5a illustrates colorimetric “naked eye” sensing using polypyrrole magnetic nanoparticles (Ppy NP) of the present invention to which an HRP and anti-EpCAM mixture (HRP-loaded/anti-EpCAM) is bound;

FIGS. 5b and 5c illustrate colorimetric “naked eye” sensing and UV-vis absorption spectrum analysis results of circulating tumor cells isolated from samples from early cancer patients using polypyrrole magnetic nanoparticles (Ppy NP) of the present invention to which an HRP and anti-EpCAM mixture (HRP-loaded/anti-EpCAM) is bound;

FIG. 6a illustrates that EGFR Exon 21 L858R gene mutation detected from cancer tissues of patients is the same as that in CTCs isolated from blood of cancer patients, using antibody mixture-bound polypyrrole magnetic nanowires (Ab mixture_mPpyNWs) according to the present invention; and

FIG. 6b illustrates EGFR Exon 21 L858R gene mutation in circulating tumor cells (CTCs) isolated from blood of cancer patients, investigated using digital PCR.

### DETAILED DESCRIPTION OF EXEMPLARY EMBODIMENTS

The term “circulating tumor cells,” as used in the present invention refers to cells which are detected in a process in which some tumor cells detached from primary tumors are introduced into blood vessels or lymphatic vessels and migrate into other tissues or organs.

The type of “cancer cells” used in the present invention is not specifically limited and the circulating tumor cells of the present invention may be, without being limited to, circulating tumor cells (CTCs) or circulating tumor stem cells (CTSCs).

In addition, the type of “cancer” used in the present invention is not specifically limited and examples of the cancer include liver cancer, colorectal cancer, rectal cancer, endometrial carcinoma, ovarian cancer, renal pelvic cancer, pancreatic cancer, carcinoma of the small intestine, hepatopancreatobiliary cancer, gastric or stomach cancer, brain tumors, breast cancer, and the like.

The type of “antibody” used in the present invention is not specifically limited and examples thereof may include anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

In addition, the antibody of the present invention may be an antibody mixture and the antibody mixture of the present invention may include anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

The antibody mixture may also further include horseradish peroxidase (HRP), but the present invention is not limited thereto.

In addition, the conductive polymer of the present invention may be, without being limited to, polyacetylene, polypyrrole, polythiophene, poly(3,4-ethylenedioxythiophene) (PEDOT) polyaniline, or a derivative thereof.

The conductive polymer of the present invention may be, without being limited to, a nanostructure including a conductive polymer on which a large amount of magnetic nanoparticles is loaded and to which an antibody, to which biotin is attached, is bound.

The nanostructure of the present invention may also be a nanowire, nanorod or nanoparticle. Preferably, the nanostructure may be a nanowire, but the present invention is not limited thereto.

In addition, since a large amount of magnetic nanoparticles are loaded on the nanostructure of the present invention, the nanostructure has a larger transverse relaxation rate (R₂) than nanoparticles at the same iron (Fe) concentration. More particularly, the magnetic nanowire of the present invention may have a transverse relaxation rate of 20 to 60 mMFeS⁻¹ and a saturation magnetization value of −90 to 90 emu/g, but the present invention is not limited thereto.

In addition, the nanowire of the present invention may have a diameter of 100 nm to 300 nm, a length of 5 μm to 30 μm, and an average length of 17 μm, but the present invention is not limited thereto.

In another aspect of the present invention, the present invention provides a method of detecting and isolating circulating tumor cells. More particularly, the method may include (1) a step of treating a subject sample with the nanostructure of the present invention; and (2) a step of detecting circulating tumor cells from the nanostructure using a magnetic field generated by a magnet. In addition, the method may further include a step of isolating the circulating tumor cells from the nanostructure using a compound, but the present invention is not limited thereto.

In the method of detecting and isolating circulating tumor cells according to the present invention, the compound may be glutathione, but the present invention is not limited thereto. The compound may be any material that can cleave a disulfide bond.

In another aspect of the present invention, the present invention provides a method of colorimetrically detecting circulating tumor cells. More particularly, the method may include (1) a step of treating a subject sample with the nanostructure according to the present invention, wherein the nanostructure further includes horseradish peroxidase (HRP) and (2) a step of determining a color of the nanostructure with the naked eye. The method may further include a step of quantifying a concentration of circulating tumor cells in the subject sample by measuring a change in the color of the nanostructure by means of a spectrometer or colorimeter.

In the present invention, the sample may be blood, but the present invention is not limited thereto.

In another aspect of the present invention, the present invention provides a kit for diagnosing cancer including the nanostructure for detecting and isolating circulating tumor cells. More particularly, the kit may be a biosensor, but the present invention is not limited thereto.

In another aspect of the present invention, the present invention provides a method of providing information for diagnosing the onset and/or prognosis of cancer. More particularly, the method may include a step of extracting or isolating DNA from circulating tumor cells detected with the nanostructure for detecting and isolating circulating tumor cells according to the present invention and analyzing the DNA. In the analyzing, the concentration, copy number, or a nucleotide sequence of DNA in a sample are analyzed to determine gene mutations therein.

In another aspect of the present invention, the present invention provides a method of diagnosing cancer using the nanostructure for detecting and isolating circulating tumor cells.

More particularly, the method may include a step of extracting or isolating DNA from circulating tumor cells detected with the nanostructure for detecting and isolating circulating tumor cells of the present invention and analyzing the DNA. In the analyzing, the concentration, copy number, or a nucleotide sequence of DNA in a sample are analyzed to determine gene mutations therein.

Now, the present invention will be described in more detail with reference to the following preferred examples. These examples are provided for illustrative purposes only and should not be construed as limiting the scope and spirit of the present invention.

### EXAMPLES

### Example 1. Manufacture of Antibody Mixture-Bound Polypyrrole Magnetic Nanostructure

1-1. Manufacture of Antibody Mixture-Bound Polypyrrole Magnetic Nanoparticles (Ab Mixture_mPpyNPs)

For the synthesis of hyaluronic acid (HA)-conjugated Ppy NPs, 0.125 g of PVP (M.W 29,000) were dissolved in 3.125 mL of ultrapure water and then vigorously stirred at room temperature for 30 min. Subsequently, 16.25 mL of pyrrole and 1.5 mL of 10-nm magnetic NPs were added with gentle stirring. After 10 min, 125 μL of iron (II) chloride hexahydrate (0.75 g/mL) and 100 mg of HA (40K) were quickly added and allowed to polymerize for 3 h at room temperature (RT). The products were purified by dialysis for 2 days, and then the purified solution was freeze-dried and stored in a vacuum until use. Next, approximately 2 mg of HA-Ppy NPs were mixed in 1 mL of 0.4 M EDC and 0.1 M NHS for 45 min and then centrifuged at 17,000 rpm. Finally, HA-Ppy NPs were resuspended in 1 mL of 10 μL/mL of streptavidin to conjugate a 10 μL/mL biotinylated antibody mixture (EpCAM, EGFR, N-cadherin, TROP-2, vimentin). The resulting solution was centrifuged again at 17,000 rpm and stored in 1×PBS until use.

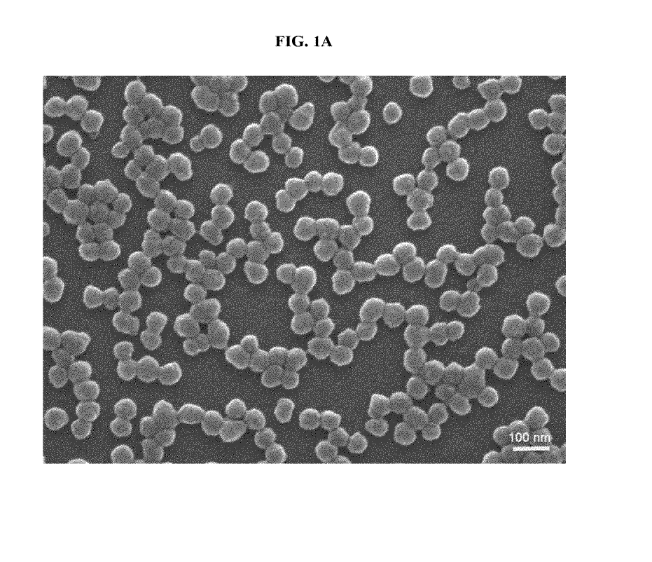

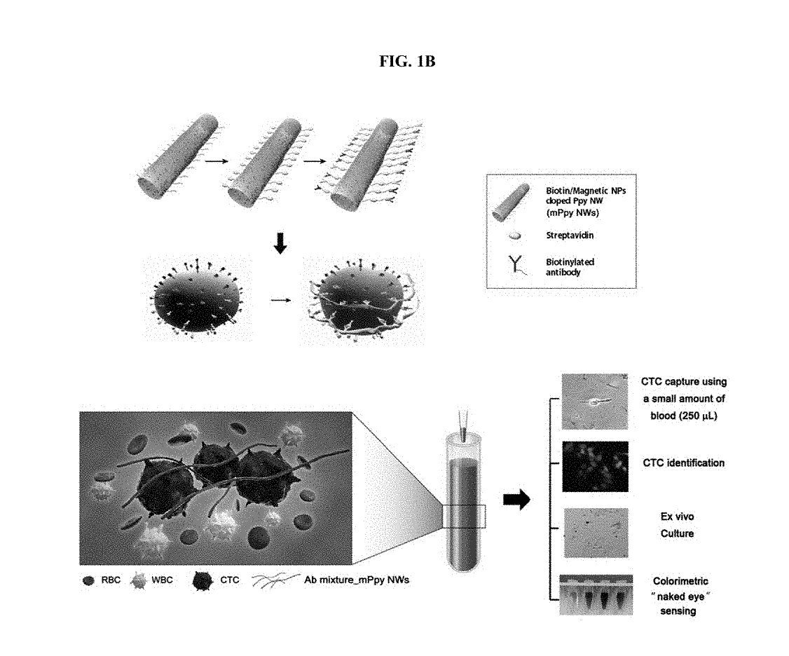

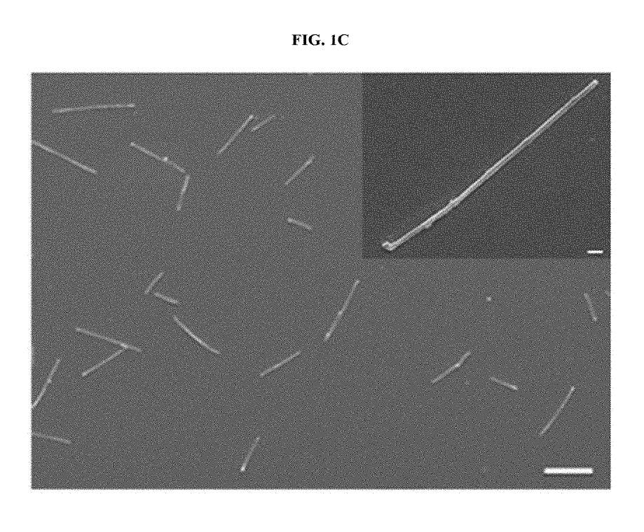

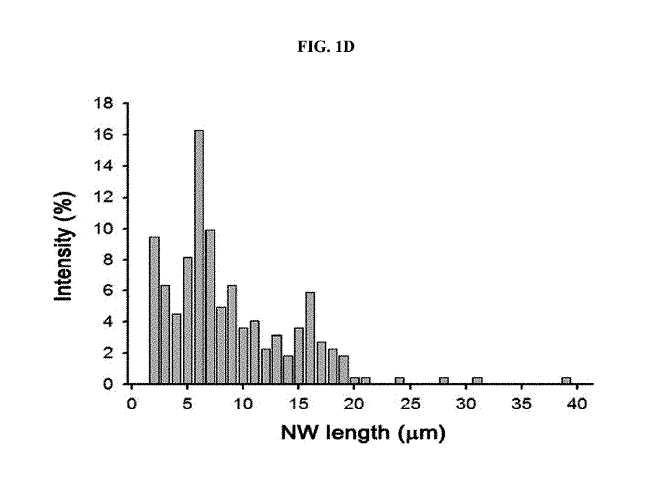

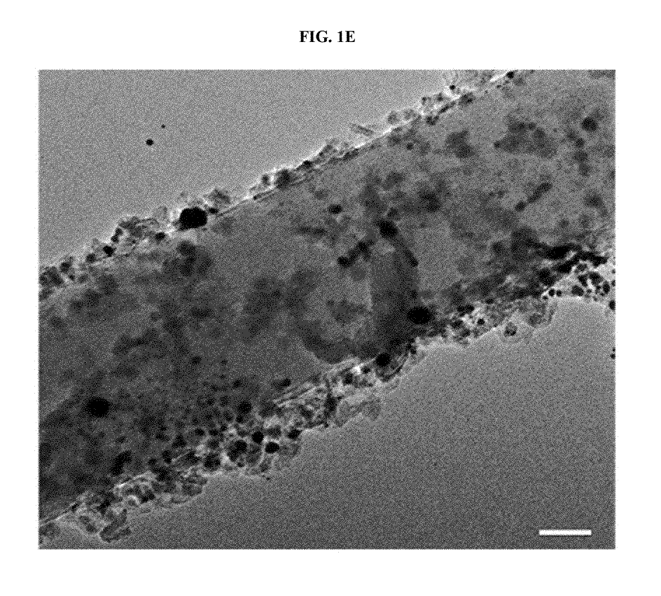

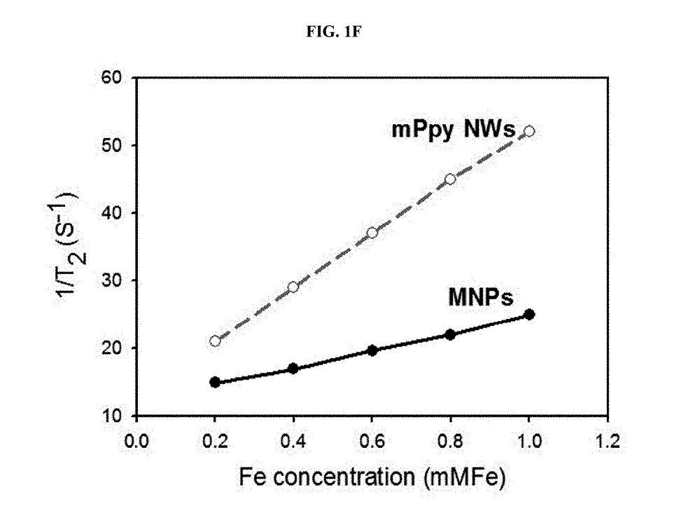

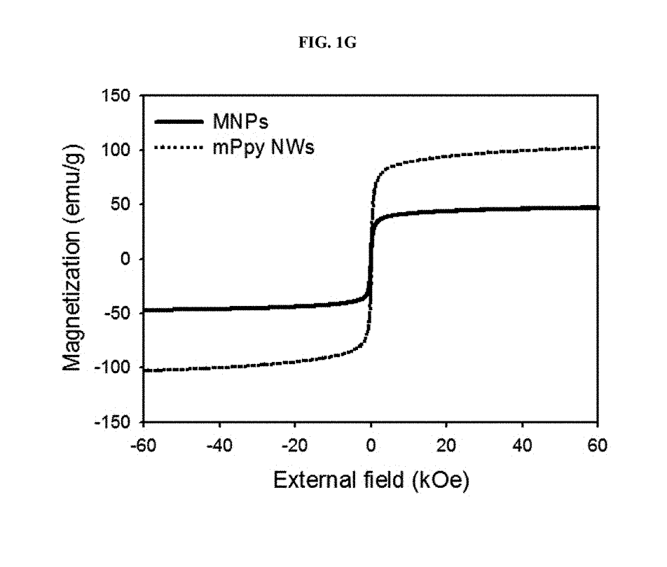

As a result, it was confirmed that, as illustrated in FIG. 1A, it was confirmed that antibody mixture-bound polypyrrole magnetic nanoparticles (Ab mixture_mPpyNPs) were manufactured and were visualized by a scanning electron microscope (SEM) image.

1-2. Manufacture of Antibody Mixture-Bound Polypyrrole Magnetic Nanowires (Ab Mixture_mPpyNWs)

An approximately 150-nm-thick Au layer was deposited on one side of the AAO template (Whatman; pore diameter, 200 nm) by a conventional thermal evaporation technique. All electrochemical experiments were performed using a potentiostat/galvanostat (BioLogic SP-150), where an Au-coated AAO template, Ag/AgCl (3.0 M NaCl type), and a platinum wire were used as working, reference, and counter electrodes, respectively. As illustrated in FIG. 1B, for the preparation of the Ab mixture_mPpyNWs, 30 μL of magnetic NPs (˜10 nm in a diameter) was dropped on top of the Au-coated AAO disc and drawn inside the AAO pores with moderate aspiration at RT. To prepare Ppy NWs doped with a high density of magnetic NPs and conjugated to five different types of antibodies, we electrochemically deposited Ppy in the pores of the AAO template in 0.01 M poly(4-styrene sulfonic acid) and 0.01 M pyrrole containing 1 mg/mL NHS-ss-biotin and applied chronoamperometry at 1.0 V (vs. Ag/AgCl) for 7 min. The resulting AAO templates were rinsed several times with ultrapure water and immersed in 2 M NaOH to obtain free-standing Ppy NWs doped with magnetic NPs and ss-biotin molecules. Subsequently, 30 mM EDC and 6 mM NHS were added to the resulting Ppy NWs to activate the carboxylic acid groups. These Ppy NWs were incubated with streptavidin (10 μg/mL) for 45 min and rinsed with water. Next, the biotinylated antibody mixture (i.e., biotinylated anti-EpCAM, biotinylated anti-EGFR, biotinylated anti-N-cadherin, biotinylated anti-TROP-2, and biotinylated anti-vimentin (10 μg/mL in PBS)) was conjugated to streptavidin-terminated Ppy NWs at 4° C. overnight to prepare the Ab mixture_mPpyNWs. The morphologies of the Ab mixture_mPpyNWs were investigated by scanning electron microscopy (JSM-6701F, JEOL), with an accelerating voltage of 15 kV and transmission electron microscope (G2F30, Tecnai) with an accelerating voltage of 300 kV. Magnetic measurements were performed at RT using a SQUID-VSM magnetometer (MPMS-VSM, Quantum Design, San Diego, Calif., USA). The applied magnetic field was varied from 70 to −70 kOe. The transverse relaxation time, T2, was analyzed using a 7 Tesla MRI instrument (Bruker BioSpin MRI GmbH, Billerica, Mass., USA; echo time [TE]=6.5 ms and repetition time [TR]=1600 ms).

As a result, it was confirmed that, as illustrated in FIG. 1C, it was confirmed that antibody mixture-bound polypyrrole magnetic nanowires (Ab mixture_mPpyNWs) were manufactured and were visualized by a scanning electron microscope (SEM) image (Scale bar 10 μm). SEM images showed the long shape of the Ab mixture_mPpyNWs with diameters of about 200 nm and an average length of about 16 μm.

In addition, as illustrated in FIG. 1D, an average length distribution of antibody mixture-bound polypyrrole magnetic nanowires (Ab mixture_mPpyNWs) were evaluated.

In addition, as illustrated in FIG. 1E, it was confirmed that, a transmission electron microscopy (TEM) image (Scale bar 50 μm) of Ab mixture_mPpyNWs revealed the presence of magnetic NPs embedded inside the NW matrix with randomly distributed and densely packed arrangements.

In addition, as illustrated in FIG. 1F, transverse relaxation rates (1/T₂, S⁻¹) of antibody mixture-bound polypyrrole magnetic nanowires (Ab mixture_mPpyNWs) and magnetic nanoparticles (MNPs) of the present invention were compared using magnetic resonance imaging contrast. As a result, Ab mixture_mPpyNWs exhibited a significant increase in magnetic resonance imaging contrast (R₂=53 mMFeS⁻¹) relative to MNPs (R₂=21 mMFeS⁻¹) at the same Fe concentration.

In addition, as illustrated in FIG. 1G saturation magnetization of antibody mixture-bound polypyrrole magnetic nanowires (Ab mixture_mPpyNWs) and magnetic nanoparticles (MNPs) of the present invention were compared. As a result, Ab mixture_mPpyNWs exhibited a significant increase in saturation magnetization (Ms=82 emu/g) relative to MNPs (Ms=45 emu/g) at the same Fe concentration. Indeed, synergistic magnetism resulting from the assembly of multiple magnetic nanoparticles in a confined geometry made nanowires more susceptible to magnetic fields by orienting the magnetic moments of each nanoparticle, enabling precise control and selective manipulation of the isolated cancer cells.

### Example 2. Evaluation of the Cell Capture Efficiency of Magnetic Nanostructures Using the Antibody Mixture

2-1. Comparison of the Cell Capture Efficiency of Between Antibody Mixture and a Single Antibody Using Magnetic Nanowires

EpCAM-positive (HCT116, MCF7) and -negative (MDA-MB-231, MIA PaCa-2) cells were purchased from the American Type Culture Collection (ATCC), grown in Dulbecco's modified Eagle's medium (DMEM) or Roswell Park Memorial Institute (RPMI)-1640 medium supplemented with 10% fetal bovine serum (FBS) and 100 units/mL penicillin/streptomycin, and maintained in a humidified incubator with 5% CO₂ at 37° C. Cell culture reagents were purchased from Thermo Scientific, Hyclone, and Gibco. To compare of the capture efficiency of between antibody mixture and a single antibody using magnetic nanowires, antibody mixture-bound polypyrrole magnetic nanowires were prepared, as described in Example 1-2. To evaluated the cell capture performance of the Ab mixture_mPpyNWs using four different cell lines, including EpCAM-positive (HCT-116, MCF7) and -negative (MIA PaCa-2, MDA-MB-231) cell lines, that were spiked into 0.1% PBS/BSA or peripheral blood samples from healthy donors. Initially, the Ab mixture_mPpyNWs were incubated in cell suspensions with different numbers of cells (3˜100 cells/mL), followed by gentle shaking for 30 min at RT to induce attachment of the target cells to the NWs. Subsequently, a magnetic field created by a magnet was used on the sample tubes (1.5 mL microcentrifuge tubes) to efficiently separate the captured cells. The magnetic separation was conducted with the MagneSphere® Technology Magnetic Separation Stands (Promega, USA) that contain the samarium/cobalt magnet, with energy products (BHₘₐₓ) ranging from 16 to 33 megagauss-oersteds (MGOe), which is approximately equivalent to 128 to 264 kJ/m³.

After removal of the supernatant, the collected cell complex was washed with 1×PBS, resuspended in RPMI-1640 medium, and transferred to a cover glass in a 6-well plate. To evaluate the captured cells, immunofluorescence staining with dye-conjugated antibodies, such as FITC-anti-EpCAM, Cy3-conjugated anti-CD44, and Alexa 680-conjugated anti-CD45 was performed. For the preparation of dye-conjugated antibodies, antibody and NHS dye (antibody:NHS dye=1:2 to 1:8 molar ratio) were dissolved in 50 μL of antibody and added to 1×PBS up to 300 μL. Then, the mixture was shook gently for 1 hour at RT under dark condition. To remove unreacted NHS dye, the solution was desalted using PD Minitrap G-25 (GE Healthcare, 17-0851-01), then concentrated by Amicon Ultra Centrifugal Filters-30K (Millipore, UFC 503024) and held at 4° C. before use. Subsequently, after seeding the captured cells onto the cover glass, 0.1 μM of fluorescent dye-conjugated antibodies (FITC-anti-EpCAM, Cy3-conjugated anti-CD44, and Alexa 680-conjugated anti-CD45) was added to the cell medium and held in a 5% CO₂ incubator at 37° C. The immobilized cells were also stained with 4′,6-diamidino-2-phenylindole (DAPI) to identify the nucleus and rinsed with PBS several times. Labeled cells were examined under a Zeiss LSM 710 ConfoCor 3 fluorescence microscope.

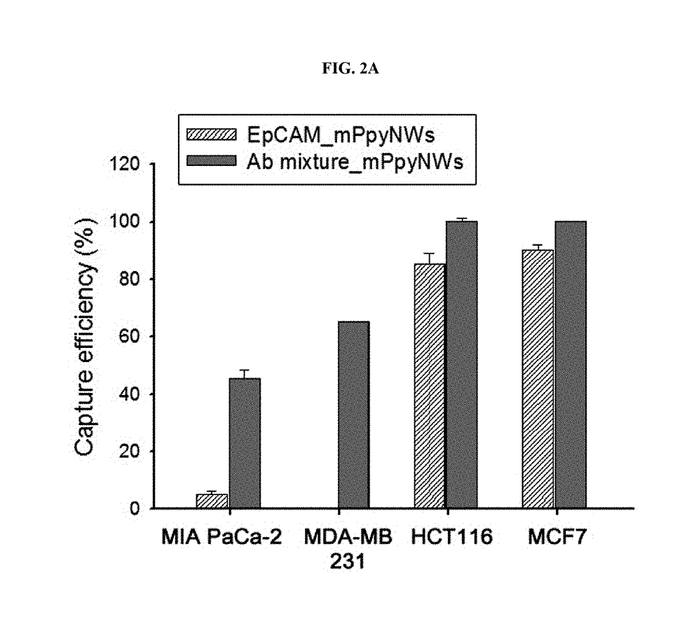

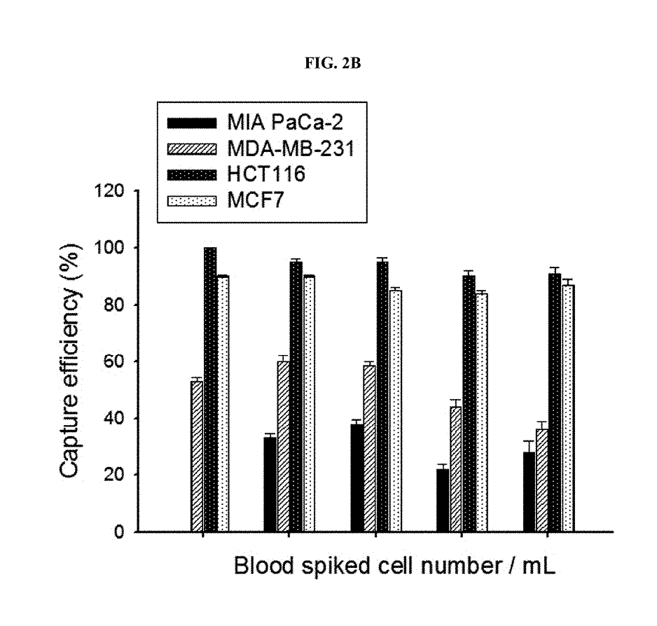

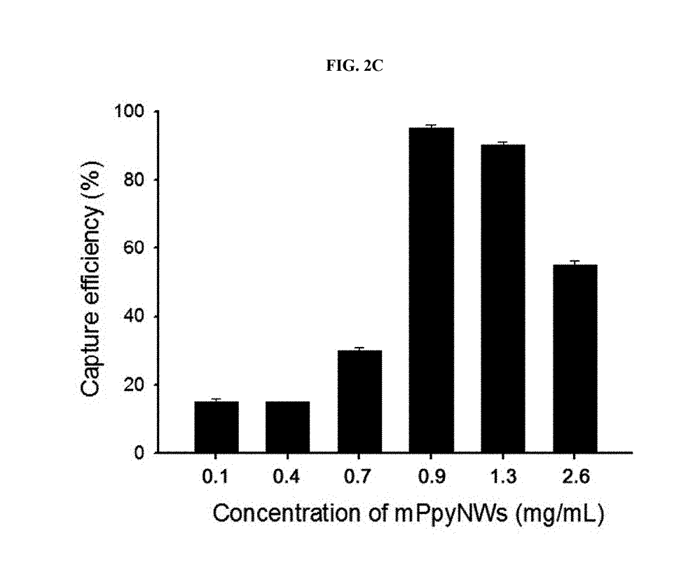

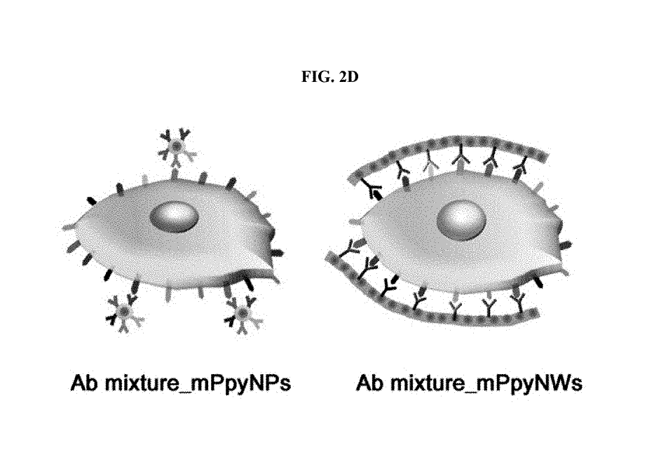

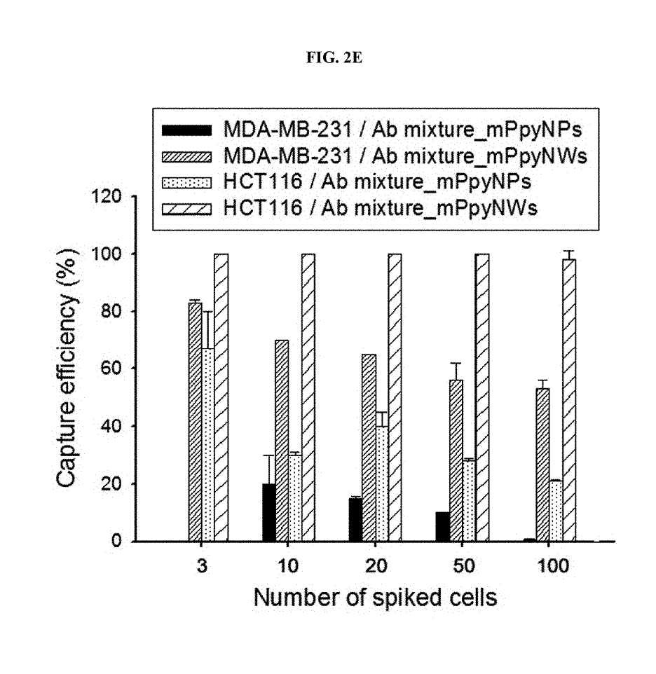

As illustrated in FIG. 2A, it was clearly confirmed that significantly higher capture efficiency was achieved using Ab mixture_mPpyNWs, regardless of the EpCAM status of tumor cells. On the other hand, the EpCAM-only approach (EpCAM_mPpyNWs) yielded a maximum capture efficiency of ˜83% for MCF7 cells, but very limited efficiency in the isolation of cell lines with nonepithelial characteristics.

In addition, as illustrated in FIG. 2B, it was confirmed that Ab mixture_mPpyNWs can greatly increase adhesion to cancer cells with different phenotypes and numbers by creating multivalent interactions and recognition between nanowires labeled with multiple types of antibodies and cell-surface receptors.

2-2. Determination of the Effective Concentration of the Nanowires for the Highest Cell Capture Efficiency

To determine of concentration of the nanowires for the highest cell capture efficiency, HCT 116 cells were spiked at concentration of 20 cells/mL in 0.1% BSA/PBS using antibody mixture-bound polypyrrole magnetic nanowires (Ab mixture_mPpyNWs) which were prepared as described in Example 1-2. The capture efficiency of the spiked HCT 116 cells using various concentrations of nanowires was evaluated.

As illustrated in FIG. 2C, the effective concentration of the nanowires influencing the capture efficiency of HCT 116 was determined. FIG. 2C shows a maximum yield of 0.9 mg/mL with a capture efficiency of 96% for target cancer cells, followed by a gradual decrease that was most likely a result of agglomeration and entanglement of the nanowires.

2-3. Comparison of the Capture Efficiency of Between Magnetic Nanoparticles and Magnetic Nanowires

To evaluate the capture efficiency using direct interactions between cells and nanomaterials, cells were treated as described in Example 1-2. Ab mixture_mPpyNWs with two different cell lines (EpCAM-positive HCT116 cells and EpCAM-negative MDA-MB-231 cells) were compared their performance with those of the nanoparticles.

As illustrated in FIGS. 2D and 2E, the nanowire-based approach was found to have a significant impact on the isolation of target cells relative to spherical nanoparticles. Also, it was confirmed that the elongated structure of the nanowires can provide substantial benefits by offering more available sites to accommodate a sufficient amount of antibodies, readily promoting multiple interactions with specific cancer cells and thus conferring greater sensitivity in CTC capture.

### Example 3. Evaluation of the Cell Capture Efficiency in Blood of Breast Cancer Patient Using Magnetic Nanowires

3-1. Immunofluorescence of the Captured Cell

To evaluate of the cell capture efficiency of antibody mixture-bound polypyrrole magnetic nanowires (Ab mixture_mPpyNWs), CTCs were isolated in patients with non-metastatic early breast cancer. Whole blood was collected in Vacutainer tubes containing the anti-coagulant EDTA, following procedures approved by the National Cancer Center Institutional Review Board. For the clinical application, blood samples were collected from 18 healthy volunteers and 29 patients with early-stage breast cancer. Having validated and optimized the capture conditions of Ab mixture_mPpyNWs using the artificial blood samples, their utility was demonstrated in CTC isolation from early-stage breast cancer patients. While most CTC studies require 5-10 mL of blood, the proposed NW-based approach is capable of detecting and identifying many more CTCs, even with small amounts of blood. A total of 29 blood samples from cancer patients were examined. The majority of patients had early localized breast cancer (stage I and II); however, 6 out of the 29 individuals had received adjuvant chemotherapy before surgery. Samples of 250 μL-1 mL of unprocessed blood were used for CTC isolation and analysis. In addition, CTC detection were evaluated in blood from 18 healthy donors, using 250 μL-1 mL of peripheral blood as a control. After confirming the cell capture, as described in Example 2-1, the captured cells were transferred onto coverslips in plates, and then the cells were fixed with 3.7% PFA for 15 min, permeabilized with 0.3% Triton X-100 for 10 min, and incubated in 5% BSA/PBS blocking solution for 30 min. Subsequently, the anti-EpCAM, anti-CD44, anti-vimentin, and anti-CD45 antibodies were incubated on coverslips for 90 min. Next, Alexa Fluor 488-conjugated (Invitrogen, Carlsbad, Calif., USA; green signal for EpCAM) or Alexa Fluor 647-conjugated (Invitrogen; red signal for CD44, vimentin, and CD45) secondary antibody was added to the coverslips. After 40 min, the cells were stained with Hoechst 33342 (Invitrogen; blue signal for the nucleus) and rinsed with PBS. Labeled cells were analyzed under a LSM501 META confocal microscope (Carl Zeiss, Oberkochen, Germany). In immunocytochemistry (ICC) images of captured CTCs, cells were stained with DAPI (nucleus; blue), CD45 (hematopoietic; red), EpCAM (epithelial; green), and CD44 or vimentin (mesenchymal; red). DAPI, anti-EpCAM, anti-CD44, anti-vimentin, and anti-CD45 were employed to differentiate CTCs from surrounding leukocytes and experiments were performed in quintuplicate.

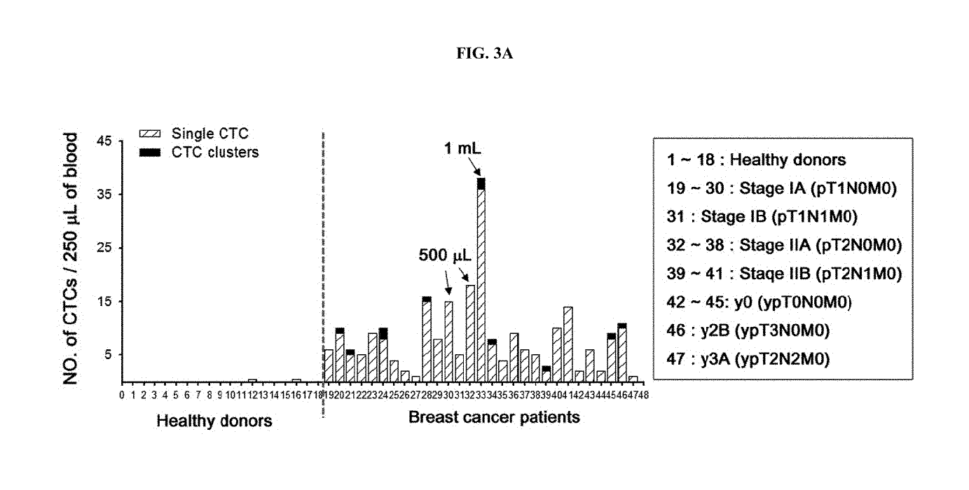

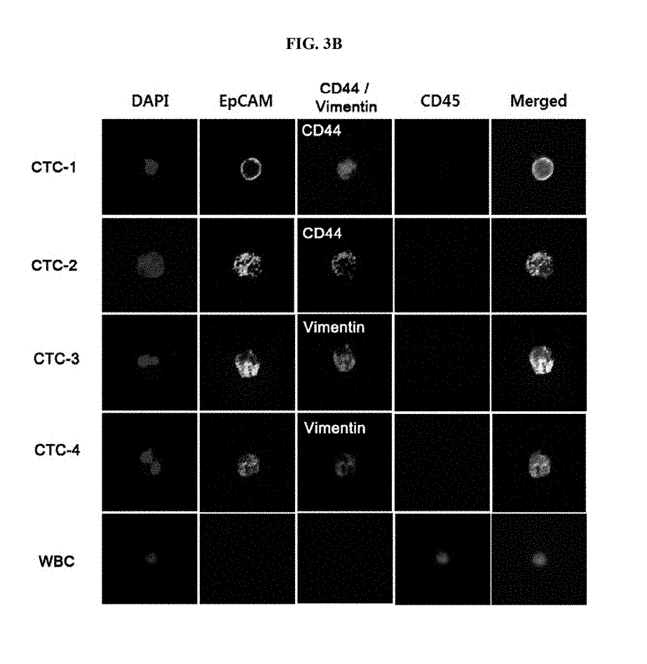

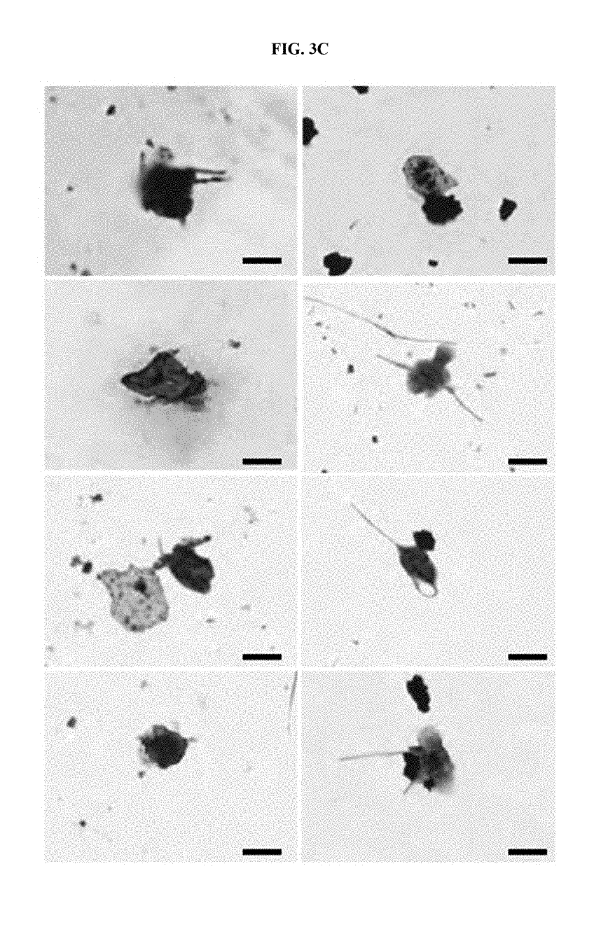

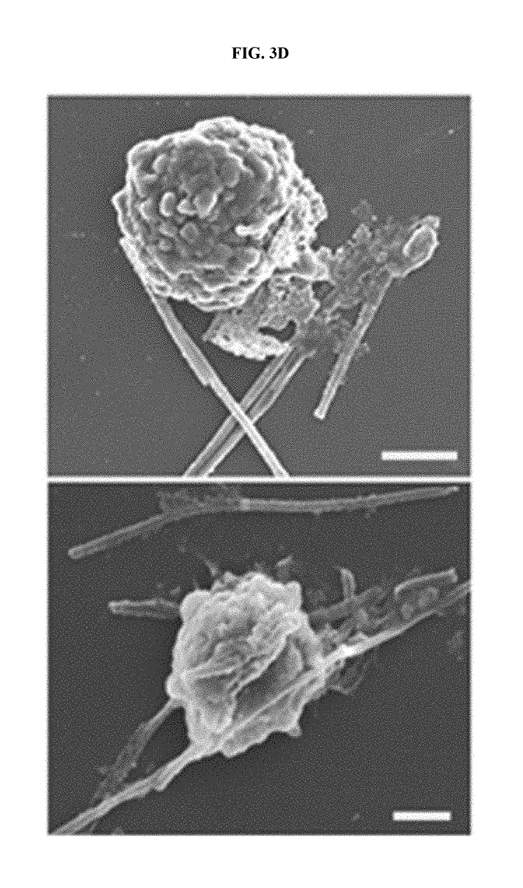

As illustrated in FIG. 3A, CTCs were identified in all blood samples of cancer patients analyzed. Also, it was confirmed that the number of leukocytes that bound non-specifically was low (<5 WBCs/250 μL of blood), indicating that Ab mixture_mPpyNWs are highly selective in capturing target CTCs and are very efficient in eliminating WBCs. Using Ab mixture_mPpyNWs, CTCs were successfully isolated from the blood of non-metastatic early-stage breast cancer patients with blood sample volumes as low as 250 μL. Interestingly, the number of CTCs isolated consistently increased with an increasing volume of blood, from 250 μL to 1 mL. Among healthy donors, 16 out of 18 showed no identifiable CTCs; however, 1 to 2 cells per 1 mL of blood were detected in 2 healthy donors.

In addition, as illustrated in FIG. 3B, CTCs were classically defined based on phenotypic expression of epithelial origin markers (DAPI+/EpCAM+/CD45− expression), whereas WBCs were defined based on the display of DAPI+/EpCAM−/CD45+. Notably, a significant number of CTCs were co-expressed with epithelial and EMT markers (e.g., CD44 or vimentin), indicating that the majority of CTCs captured were metastatically competent.

3-2. Immunohistochemistry of the Captured Cell

After confirming the cell capture, as described in Example 2-1, Immunohistochemistry was performed. Captured cells were further confirmed by additional IHC analysis after staining with the epithelial marker EpCAM and counterstaining with hematoxylin. Also, using the SuperPicture 3^{rd} Gen IHC detection kit from Invitrogen according to the manufacturer's instructions. The cells were mounted on glass slides and scanned at 400× magnification using an Olympus BX52 microscope (Tokyo, Japan) linked with image analysis software (Aperio ImageScope, Leica Biosystems, Wetzlar, Germany).

As a result, as illustrated in FIG. 3C, it was confirmed that CTCs were captured. Cancer cells appear to be brown as a result of the DAB-substrate reaction. Nuclei are stained blue because of hematoxylin counterstaining.

3-3. Scanning Electron Microscope Images of CTCs

To analyze morphologies of CTCs that were captured by the Ab mixture_mPpyNWs, scanning electron microscope images of circulating tumor cells collected from blood of early-stage breast cancer patients using antibody mixture-bound polypyrrole magnetic nanowires (Ab mixture_mPpyNWs) were observed by SEM (JSM-6701F, JEOL) with an accelerating voltage of 5 kV. Briefly, the captured cells were fixed with 3.7% PFA for 2 h and exposed to ethanol in increasing concentrations (50%, 70%, 90%, and 100%) for 15 min each for dehydration, followed by complete air drying. The samples were sputter-coated with platinum gold before examination by SEM.

As illustrated in FIG. 3D, scanning electron microscope images (Scale bar, 5 μm) of circulating tumor cells collected from blood of early-stage breast cancer patients were confirmed.

### Example 4. Evaluation of the Glutathione-Mediated Retrieval Using Magnetic Nanowires

To retrieve CTCs attached Ab mixture_mPpyNWs, the captured cells were easily released by treatment with 50 mM glutathione (GSH) solution, with mild shaking at 500 rpm for 60 min. Then, the released cells were seeded onto a 24-well plate to monitor cell growth and proliferation.

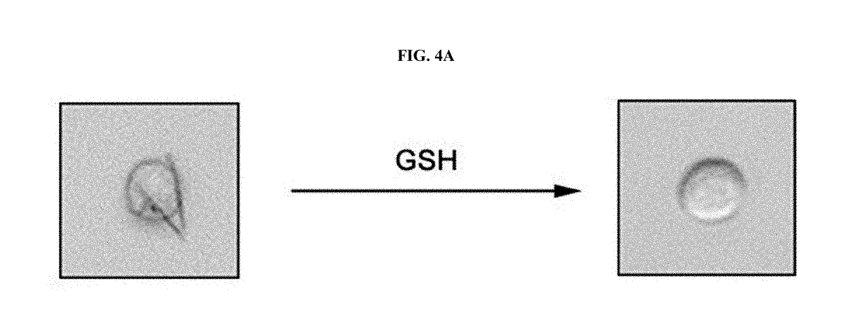

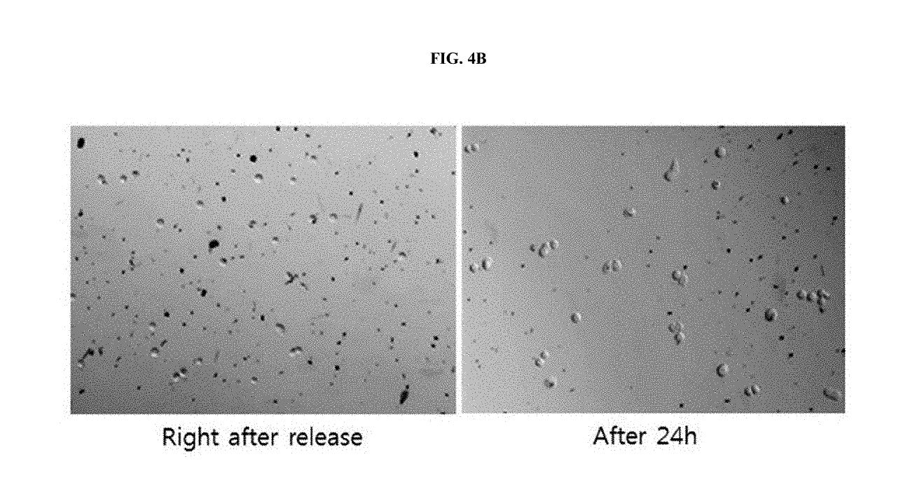

As a result, as illustrated in FIG. 4A, it was confirmed that the ss-biotin moieties as dopants inside the individual nanowires enabled cell release without any cell damage. Indeed, it is possible to achieve GSH-mediated release of CTCs by breaking the disulfide bonds of ss-biotin that facilitate the retrieval of captured cells from the Ab mixture_mPpyNW.

In addition, as illustrated in FIG. 4B, 24 h after cells were released from nanowires, cellular growth and proliferation were observed. Also, it was confirmed that GSH-mediated treatment did not influence cell viability and, thus, their applicability for use in downstream subculture or molecular analyses.

### Example 5. Evaluation of the Colorimetric “Naked Eye” Sensing of the Captured Cancer Cells

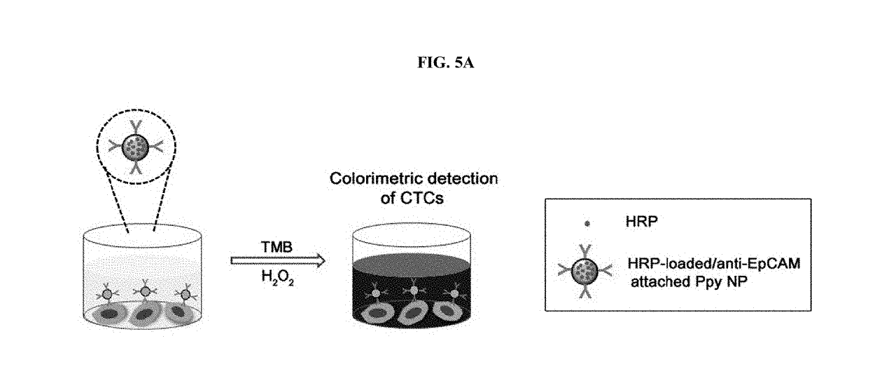

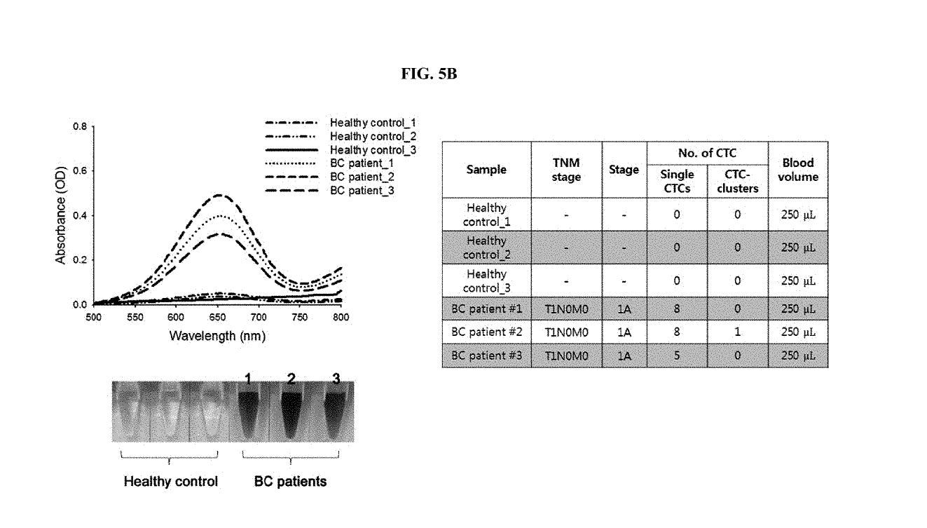

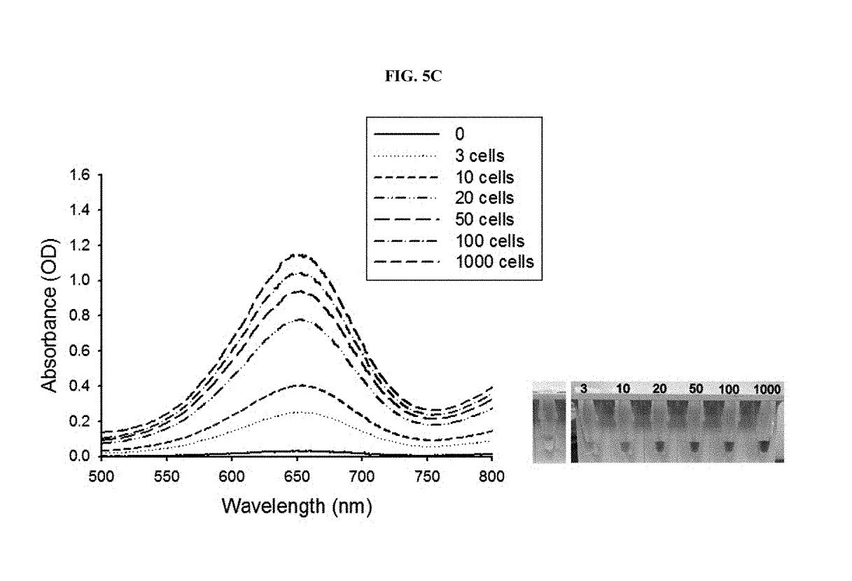

As described in Example 3, captured cells were subsequently fixed and immunostained with representative epithelial markers to confirm the presence of CTCs by comprehensive image analysis; however, this involved complex, time-consuming, and laborious procedures to obtain results. In the present invention, a rapid and reliable strategy for in situ “naked eye” detection to predict the presence of cancer cells via a simple colorimetric immunoassay was adapted as illustrated in FIG. 5A.

To evaluate of the colorimetric “naked eye” sensing of the captured circulating tumor cells from samples from early cancer patients, approximately 2 mg of HA-Ppy NPs was mixed with 1 mL of 0.4 M EDC and 0.1 M NHS for 45 min, and then the solution was centrifuged to remove excess chemicals at 17,000 rpm. Next, 1 mg of HRP and 20 μg of biotinylated anti-EpCAM (weight ratio of HRP:anti-EpCAM=50:1) was added to the dispersion described above under ultrasonic vibration at 4° C. overnight. The unreacted reagents were removed by gel filtration using a PD10 column (GE Healthcare). For in vitro colorimetric measurement, cells were seeded into 96-well plates at densities of 0 cells/mL, 3 cells/mL, 10 cells/mL, 20 cells/mL, 50 cells/mL, 102 cells/mL, and 103 cells/mL, where approximately 0.15 mg of HRP-loaded/anti-EpCAM-attached PpyNPs was added, and then plates were held for 10 min in a 5% CO₂ humidity incubator at 37° C. Following three washes in 1×PBS, 100 μL of 10 mM TMB, 100 μL of 0.1 M H₂O₂, and 8004 of 0.2M sodium acetate buffer (pH5.0) were added to the dispersion described above and held in the dark for 3 min at RT. To determine the correlation between the numbers of captured cells and the absorbance, UV-Vis detection was conducted at a wavelength of 652 nm, using a DU 730 UV-Vis spectrophotometer (Beckman Coulter, USA). For clinical samples, the captured cells from healthy donors or breast cancer patients were transferred into 6-well plates and the same procedure using UV-vis spectroscopy was applied.

As illustrated in FIG. 5B, after colorimetric TMB substrate was added to the cell suspension that immediately triggered a reaction, ultimately yielding a color signal proportional to the number of cancer cells captured on the NWs. As an accurate and cost-effective pre-assessment method, a discernible color change can not only directly indicate the presence of CTCs in the blood samples of patient but also allow non-detrimental retrieval of viable cells after assay. Notably, there were nearly no changes in the absorbance values in the blood of healthy individuals. Indeed, the absorbance at 652 nm accompanying the color change dramatically increased with an increase in the number of captured CTCs, showing the assay to be sufficiently sensitive and selective for the detection of cancer cells.

### Example 6. Evaluation of Gene Mutation in CTCs Isolated from Blood of Cancer Patients

To confirm EGFR Exon 21 L858R gene mutation in circulating tumor cells (CTCs) isolated from blood of cancer patients, sample were investigated using a PCR amplification method. And then digital PCR was carried out to compare detection frequencies of EGFR Exon 21 L858R mutations in the patient blood sample with tissue sample.

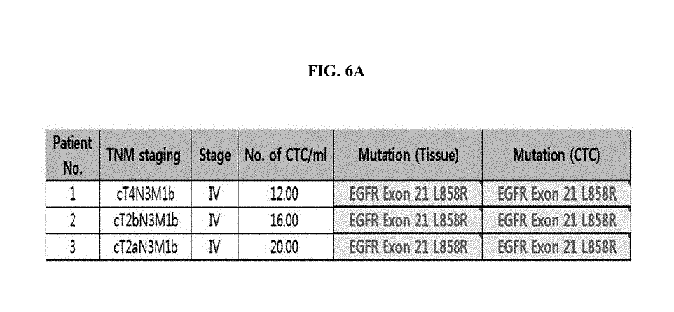

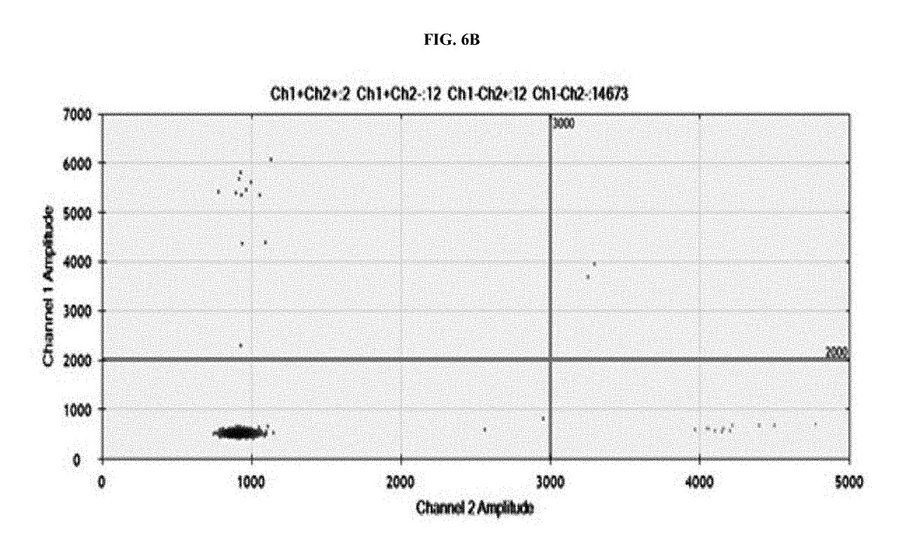

As illustrated in FIG. 6A, it was confirmed that EGFR Exon 21 L858R gene mutation detected from cancer tissues of patients is the same as that in CTCs collected from blood of cancer patients, using antibody mixture-bound polypyrrole magnetic nanowires (Ab mixture_mPpyNWs).

In addition, as illustrated in FIG. 6B, it was confirmed that EGFR Exon 21 L858R gene mutation detected from cancer blood of patients No. 3 was investigated using digital PCR.

As described above, the magnetic nanostructure according to the present invention comprising a conductive polymer, to which an antibody and magnetic nanoparticles are bound, allows effective detection of circulating tumor cells from early stage cancer patients and various circulating tumor cell types using a small amount of blood and monitoring of circulating tumor cells with the naked eye through colorimetric detection. In addition, a very small amount of circulating tumor cells present in blood can be efficiently captured with a strong magnetic field generated by a large amount of the loaded magnetic nanoparticles. Further, in detecting, isolating, and collecting the circulating tumor cells in a very small amount, a long nanowire structure and various antibody types are used, whereby contact with cancer cells may be increased and strong bonding may be formed. In addition, sensitivity is increased and various interactions with cancer cells are facilitated, thereby exhibiting remarkably increased detection and isolation effects compared to conventional technology. Therefore, the magnetic nanostructure according to the present invention is anticipated to be utilized in extracting DNA from circulating tumor cells to diagnose gene mutations, as well as early cancer diagnosis and treatment.

The aforementioned description of the present invention is provided by way of example and those skilled in the art will understood that the present invention can be easily changed or modified into other specified forms without change or modification of the technical spirit or essential characteristics of the present invention. Therefore, it should be understood that the aforementioned examples are only provided by way of example and not provided to limit the present invention.

What is claimed is:

1. A nanostructure for detecting and isolating circulating tumor cells comprising a conductive polymer, wherein an antibody is bound to the conductive polymer and magnetic nanoparticles are loaded onto the conductive polymer.

2. The nanostructure according to claim 1, wherein the antibody is one or more selected from the group consisting of anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

3. The nanostructure according to claim 1, wherein the antibody is an antibody mixture comprising anti-epithelial cell adhesion molecule (anti-EpCAM), anti-epidermal growth factor receptor (anti-EGFR), anti-N-cadherin, anti-trophoblast cell-surface antigen (anti-TROP2), and anti-vimentin.

4. The nanostructure according to claim 3, wherein the antibody mixture further comprises horseradish peroxidase (HRP).

5. The nanostructure according to claim 1, wherein the conductive polymer is polyacetylene, polypyrrole, polythiophene, poly(3,4-ethylenedioxythiophene) (PEDOT), polyaniline, or a derivative thereof.

6. The nanostructure according to claim 1, wherein the nanostructure is a nanowire, nanorod, or nanoparticle.

7. The nanostructure according to claim 1, wherein the circulating tumor cells are circulating tumor cells (CTCs) or circulating tumor stem cells (CTSCs).

8. The nanostructure according to claim 7, wherein the circulating tumor cells are circulating tumor cells (CTCs).

9. A method of detecting and isolating circulating tumor cells, the method comprising:
treating a subject sample with the nanostructure according to claim 1; and
detecting circulating tumor cells from the nanostructure using a magnetic field generated by a magnet.

10. The method according to claim 9, further comprising isolating the circulating tumor cells from the nanostructure using a compound.

11. The method according to claim 10, wherein the compound is glutathione.

12. The method according to claim 9, wherein the sample is blood.

13. A method of colorimetrically detecting circulating tumor cells, the method comprising:
treating a subject sample with the nanostructure according to claim 1, wherein the nanostructure further comprises horseradish peroxidase (HRP); and
determining a color of the nanostructure with the naked eye.

14. The method according to claim 13, further comprising quantifying a concentration of circulating tumor cells in the subject sample by measuring a change in the color of the nanostructure by means of a spectrometer or colorimeter.

15. The method according to claim 13, wherein the sample is blood.

16. A kit for diagnosing cancer, the kit comprising the nanostructure according to claim 1.

17. The kit according to claim 16, wherein the kit is a biosensor.

18. A method of providing information for diagnosing onset or prognosis of cancer, the method comprising extracting or isolating DNA from circulating tumor cells detected by the nanostructure according to claim 1 and analyzing the DNA.

19. The method according to claim 18, wherein, in the analyzing, a concentration, a copy number, or a nucleotide sequence of DNA in a sample is analyzed to investigate gene mutations.
